(19) United States
(12) Patent Application Publication
(10) Pub. No.: US 2004/0236275 A1
(43) Pub. Date: Nov. 25, 2004

(54) Catheter having a light emitting component

(76) Inventors: Sean R. Pruitt, San Jose, CA (US); David Craig, Mountain View, CA (US)

Correspondence Address: FULWIDER PATTON LEE & UTECHT, LLP, HOWARD HUGHES CENTER, 6060 CENTER DRIVE, TENTH FLOOR, LOS ANGELES, CA, 90045, US

(21) Appl. No.: 10/441,686

(22) Filed: May 20, 2003

(51) Int. Cl. A61M 29/00

(52) U.S. Cl. 604/96.01; 264/21; 264/634; 604/523

(57) ABSTRACT

The invention is directed to a catheter component having at least a section with a light emitting substance. Preferably, the light emitting substance is in or on a polymeric wall defining a lumen and a port, so that the port is outlined by the light emitting substance. In one embodiment, the component is a shaft of the catheter, and in another embodiment, the component is a proximal adapter connected to the proximal end of the catheter shaft. The light emitting substance preferably emits visible light, to facilitate the ability to see the component as other devices or components are introduced into or attached onto the port outlined by the light emitting substance during a medical procedure.

### BACKGROUND OF THE INVENTION

This invention generally relates to medical devices, and particularly to intracorporeal devices for therapeutic or diagnostic uses, such as balloon catheters.

In percutaneous transluminal coronary angioplasty (PTCA) procedures, a guiding catheter is advanced until the distal tip of the guiding catheter is seated in the ostium of a desired coronary artery. A guidewire, positioned within an inner lumen of a dilatation catheter, is first advanced out of the distal end of the guiding catheter into the patient's coronary artery until the distal end of the guidewire crosses a lesion to be dilated. Then the dilatation catheter having an inflatable balloon on the distal portion thereof is advanced into the patient's coronary anatomy, over the previously introduced guidewire, until the balloon of the dilatation catheter is properly positioned across the lesion. Once properly positioned, the dilatation balloon is inflated with fluid one or more times to a predetermined size at relatively high pressures (e.g., greater than 8 atmospheres) so that the stenosis is compressed against the arterial wall and the wall expanded to open up the passageway. Generally, the inflated diameter of the balloon is approximately the same diameter as the native diameter of the body lumen being dilated so as to complete the dilatation but not overexpand the artery wall. Substantial, uncontrolled expansion of the balloon against the vessel wall can cause trauma to the vessel wall. After the balloon is finally deflated, blood flow resumes through the dilated artery and the dilatation catheter can be removed therefrom.

In such angioplasty procedures, there may be restenosis of the artery, i.e., reformation of the arterial blockage, which necessitates either another angioplasty procedure, or some other method of repairing or strengthening the dilated area. To reduce the restenosis rate and to strengthen the dilated area, physicians frequently implant a stent inside the artery at the site of the lesion. Stents may also be used to repair vessels having an intimal flap or dissection or to generally strengthen a weakened section of a vessel. Stents are usually delivered to a desired location within a coronary artery in a contracted condition on a balloon of a catheter which is similar in many respects to a balloon angioplasty catheter, and expanded to a larger diameter by expansion of the balloon. The balloon is deflated to remove the catheter and the stent left in place within the artery at the site of the dilated lesion.

It would be a significant advance to provide a catheter which facilitates the physician's ability to ready the catheter for use just prior to an intravascular procedure, such as a balloon dilatation or stent delivery procedure.

### SUMMARY OF THE INVENTION

The invention is directed to a catheter component having at least a section with a light emitting substance. Preferably, the light emitting substance is in or on a polymeric wall defining a lumen and a port, so that the port is outlined by the light emitting substance. In one embodiment, the component is a shaft of the catheter, and in another embodiment, the component is a proximal adapter connected to the proximal end of the catheter shaft. The light emitting substance preferably emits visible light, to facilitate the ability to see the component as other devices or components are introduced into or attached onto the port outlined by the light emitting substance during a medical procedure.

The section of the catheter having the light emitting substance is caused to glow (i.e., emit light) upon exposure to an energy source, preferably improving the visibility of the section of the catheter. Specifically, the glowing catheter section is more easily viewed by the physician in a dim or darkened cath-lab during a medical procedure. The cath-lab is typically darkened for better viewing of the fluoroscopic images of the catheter during a medical procedure. Thus, a guidewire port outlined by the light emitting substance which is glowing in the darkened cath-lab makes it easier for the physician to see the port during introduction of a guidewire into the guidewire port as part of the preparation for introducing the catheter into the patient. Similarly, a glowing section of a proximal adapter of the invention is more easily viewed, thus facilitating connecting the glowing section to, for example, a fluid source. The catheter of the invention may comprise a variety of suitable catheters including balloon catheters, guiding catheters, access catheters (e.g., femoral access sheaths), and the like.

The light emitting substance is selected from the group consisting of a luminescent substance, a phosphorescent substance, and a fluorescent substance. In a presently preferred embodiment, the light emitting substance emits visible light (i.e., light at a wavelength of about 400 nm to about 700 nm) upon exposure to an energy source. A variety of suitable energy sources can be used to cause the light emission, depending on the nature and amount of the light emitting substance. In one preferred embodiment, the energy source used to cause the light emission is visible light, such as for example, the typically minimal ambient light present in the cath-lab during the medical procedure. However, in alternative embodiments, a specifically provided energy source is used to produce the light emission, such as for example, an electron beam (e-beam), electromagnetic radiation, electric field, or ultra-violet radiation source. Suitable light emitting substances include inorganic phosphors such as zinc sulfide and strontium aluminate (phosphorescent substances), which may include activator materials if necessary and which can continue to glow a noticeable period of time (e.g., minutes) after the excitation source is extinguished.

In a presently preferred embodiment, the catheter component is formed at least in part of a blend of the light emitting substance and a polymer, although the light emitting substance may alternatively be applied as a coating on a surface of the component. Unlike coatings of the light emitting substance, blending the light emitting substance into the polymer forming the catheter component avoids the potential of coatings to come off the catheter while in the patient's body lumen. The blend preferably defines an end face of the component having a port therein, and/or an outer surface and/or and inner surface of the component. The polymer is typically melt processable, so that in one embodiment the component is formed by melt extruding the light emitting substance polymeric blend. The light emitting substance is present in a section extending along only a portion of the component at the location of the port, or alternatively it extends along the entire length of the component.

The section of the catheter component of the invention having the light emitting substance is more easily viewed, due to the glowing of the light emitting substance. Thus, in one embodiment, the speed and ease with which a guidewire is threaded into the catheter guidewire port is improved. Moreover, by extruding a blend of the light emitting substance and a polymer, the component can be readily formed with the light emitting substance in a desired component shape. These and other advantages of the invention will become more apparent from the following detailed description and exemplary drawings.

### BRIEF DESCRIPTION OF THE DRAWINGS

FIG. 1 is an elevational view, partially in section, of a balloon catheter embodying features of the invention.

FIG. 1A is an enlarged view of the distal end of the catheter of FIG. 1 taken within circle 1A.

FIGS. 2-4 are transverse cross sections of the catheter of FIG. 1 taken along lines 2-2, 3-3, and 4-4, respectively.

FIG. 5 is a longitudinal cross sectional view of a distal end of a catheter have an alternative distal tip member embodying features of the invention, having stripes of light emitting substance.

FIG. 6 is a transverse cross section of the distal tip member of FIG. 5 taken along line 6-6.

FIG. 7 is an elevational view, partially in section, an over-the-wire balloon catheter with a proximal adapter embodying features of the invention.

### DETAILED DESCRIPTION OF THE PREFERRED EMBODIMENTS

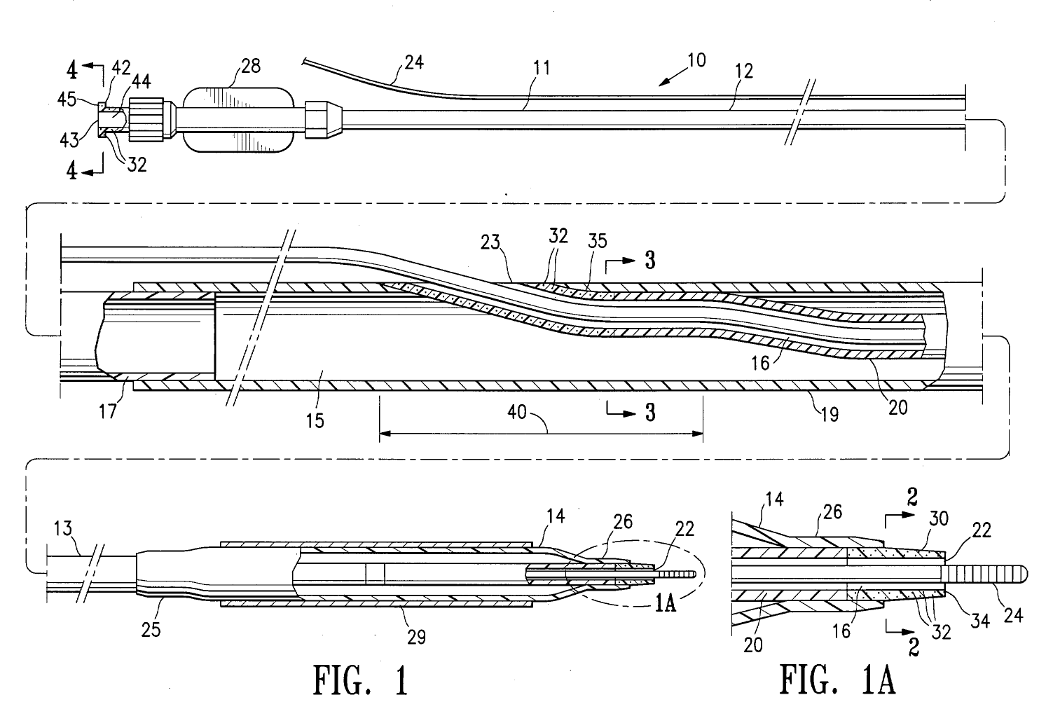

FIG. 1 illustrates a rapid exchange type balloon catheter 10 embodying features of the invention. Catheter 10 generally comprises an elongated catheter shaft 11 having a proximal end, a distal end, a proximal shaft section 12 and a distal shaft section 13, and an inflatable balloon 14 on the distal shaft section. The shaft 11 has an inflation lumen 15, and a guidewire receiving lumen 16. The proximal shaft section 12 comprises a proximal tubular member 17 defining a proximal portion of the inflation lumen 15. The distal shaft section 13 comprises an outer tubular member 19 defining a distal portion of the inflation lumen 15, and an inner tubular member 20 defining the guidewire lumen 16 extending between and in communication with a guidewire distal port 22 at the distal end of the catheter and a guidewire proximal port 23 at the proximal end of the inner tubular member 20 spaced distally from the proximal end of the shaft 11. The guidewire lumen 16 is configured to slidably receive guidewire 24 therein. Balloon 14 has a proximal skirt section 25 sealingly secured to the distal end of outer tubular member 19 and a distal skirt section 26 sealingly secured to the distal end of inner tubular member 20, so that its interior is in fluid communication with inflation lumen 15. A proximal adapter 28 at the proximal end of the catheter provides access to the inflation lumen 15.

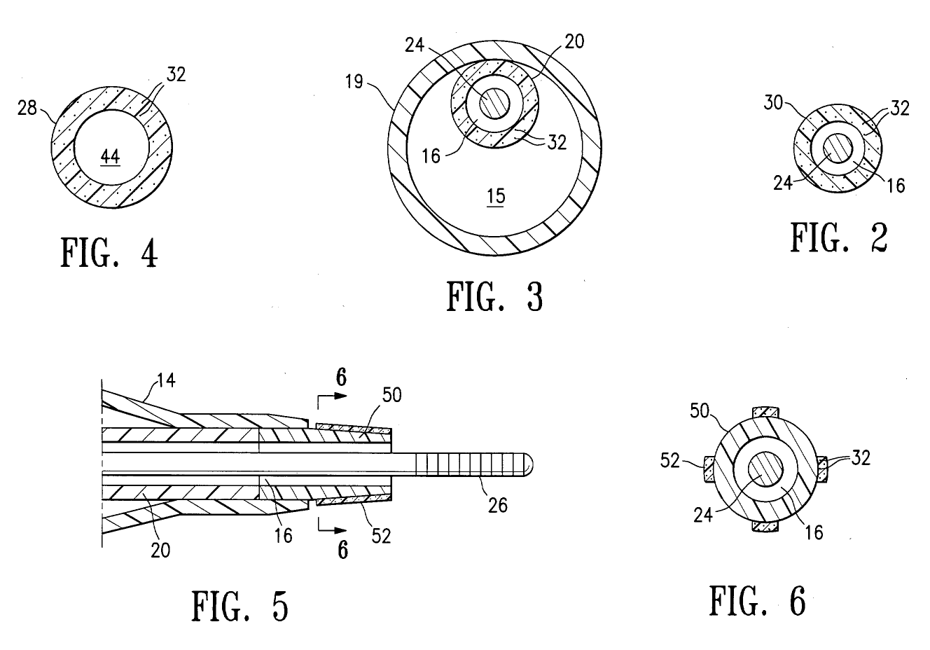

FIG. 1 illustrates the balloon 14 in a low profile configuration, prior to inflation, for introduction and advancement within the patient's body lumen 18, with a stent 29 mounted on the working length of the balloon 14. In use, the distal end of catheter 10 is advanced to a desired region of the patient's body lumen in a conventional manner either over guidewire 24 previously positioned in the body lumen, or together with guidewire 24 already in the catheter 10 guidewire lumen 16. The physician introduces guidewire 24 into the guidewire lumen 20 through distal or proximal guidewire ports 22, 23 in preparation for advancing the catheter 10 in the patient's body lumen. For example, guidewire 24 is back-loaded into guidewire lumen 20 by introducing the proximal end of the guidewire into the guidewire distal port 22 and out the guidewire proximal port 23. Alternatively, the distal end of the guidewire can be introduced into the guidewire proximal port 23 and out the distal port 22. With the distal end of the catheter advanced in the patient's body lumen into position at a stenosed region therein, balloon 14 is inflated to expand the stent 29, and the balloon deflated, and the catheter 10 removed or repositioned in the body lumen, leaving stent 29 implanted in the body lumen. Although illustrated as a stent delivery catheter in the embodiment of FIG. 1, the balloon catheter 10 of the invention may be configured to perform a variety of medical procedures including dilating a stenosis or drug delivery. Similarly, rapid exchange type catheter 10 of the embodiment of FIG. 1 may comprise a variety of suitable rapid exchange catheter shaft configurations as are conventionally known. FIG. 1A is an enlarged view of the distal end of catheter 10 of FIG. 1, taken within circle 1A. FIGS. 2 and 3 illustrate transverse cross sectional views of the catheter shaft of FIG. 1, taken along lines 2-2 and 3-3, respectively.

In the embodiment of FIG. 1, the guidewire distal port 22 and a distal section of the guidewire lumen 16 are defined by a distal tip member 30. The distal tip member 30 has a polymeric wall with a light emitting substance 32. In the illustrated embodiment, the distal tip member 30 is formed of a blend of a polymeric material and the light emitting substance 32, and the light emitting substance is dispersed throughout the wall of the distal tip member 30. The term “blend ”as used herein should be understood to refer to a polymeric mixture formed by any of a variety of methods of mixing or compounding a substance with a polymeric material. Although discussed below primarily in terms of the embodiment in which the distal tip member 30 is formed of a light emitting polymeric blend, it should be understood that in alternative embodiments the polymeric wall with a light emitting substance 32 comprises a coating of the light emitting substance 32 applied to a surface of the polymeric wall. The light emitting substance is typically a solid particulate which can be added to a polymeric material to form the blend. In a presently preferred embodiment, the distal tip member 30 is formed by hot melt extrusion using a screw extruder to form a tubular extrudate, although the blend may alternatively be molded or otherwise processed to make distal tip member 30. A variety of suitable polymeric materials are used to form distal tip member 30, and in a preferred embodiment the polymeric material is selected from the group consisting of polyamides such as nylon and copolyamides such as polyether block amide (PEBAX).

In the illustrated embodiment, the entire distal tip member 30 is formed of the light emitting polymeric blend, so that the light emitting substance is distributed throughout the length and width of the distal tip member 30. However, in alternative embodiments (not shown), only the distal end of the distal tip member 30 has the light emitting substance, or only a layer of the distal tip member 30 has the light emitting substance. In the embodiment of FIG. 1, the tip member 30 polymeric wall has a width extending from the outer surface to the inner surface of the distal tip member 30 with the entire width of the tip member polymeric wall formed of the light emitting polymeric blend. Thus, the inner surface of the distal tip member 30, defining the distal portion of the guidewire lumen 16, and the distal end surface or face 34 (oriented at a perpendicular angle to the longitudinal axis of the distal tip member) of the distal tip member 30 having the guidewire distal port 22 therein, are formed of the light emitting polymeric blend to facilitate viewing the port and lumen defining wall. In the embodiment of FIG. 1, the light emitting substance is present in a wall section extending fully around the circumference of the distal tip member 30. The distal tip member 30 generally has a length of about 0.2 to about 0.4 cm. Preferably, at least the distal most section of the distal tip member 30 has the light emitting substance, and specifically in one embodiment at least about 60% to about 70% of the length of the distal tip member 30, or about 0.12 to about 0.28 cm of the length of the distal tip member 30 has the light emitting substance. In one preferred embodiment, the light emitting substance extends along about 66% to about 100% of the total length of the distal tip member 30.

In the illustrated embodiment, the distal tip member 30 is secured to the distal end of the inner tubular member 20, and specifically is butt-joined to the distal end of the inner tubular member 20 by adhesive and/or fusion bonding. However, a variety of suitable distal tip member configurations can be used as are conventionally known. The guidewire distal port 22 outlined by light emitting substance 32 is in the distal end of the distal tip member 30 and is distal to the distal end of the balloon 14 (i.e., is distal to the distal end of the balloon distal skirt section 26 secured to the underlying section of the shaft 11).

In the embodiment of FIG. 1, the guidewire distal port 22, outlined by the light emitting substance, is defined by the distal tip member 30. However, a variety of suitable catheter configurations can be used as are conventionally known, including embodiments (not shown) in which the guidewire distal port 22 is defined by the distal end of the inner tubular member 20 or by an extended section of the balloon distal skirt section 26 extending beyond the distal end of the inner tubular member 20, instead of by a separate distal tip member 30. Thus, in accordance with an embodiment of the invention, at least the distal end section of the member defining the guidewire distal port 22 has the light emitting substance outlining the port 22, although a variety of suitable members may be used to define the guidewire distal port 22.

Similarly, the proximal end of the inner tubular member 20 defining the guidewire proximal port 23 has a polymeric wall with a light emitting substance 32 in the embodiment of FIG. 1. The proximal end face of the inner tubular member defining the port 23 has the light emitting substance, to thereby out line the port 23. In the illustrated embodiment, a proximal section 35 of the inner tubular member 20 is formed of a blend of a polymeric material and the light emitting substance 32, with the light emitting substance being dispersed throughout the wall of the proximal section 35 of the inner tubular member 20. However, in alternative embodiments (not shown), the inner tubular member 20 is a multilayered tubular member, with at least one of the layers (typically the inner layer), having the light emitting substance 32 blended into the layer or coated onto a surface of the layer. The inner tubular member 20 generally has a length of about 20 to about 40 cm. Preferably, at least a proximal-most end portion of the inner tubular member 20 has the light emitting substance, and specifically a portion having a length of about 0.2 to about 2 cm, or about 1 to about 10% of the total length of the inner tubular member 20. Thus, in the embodiment of FIG. 1, the proximal section 35 of the inner tubular member 20 formed of the light emitting polymeric blend is adjacent to a section of the inner tubular member 20 which does not have the light emitting substance, and which typically is otherwise formed of the same polymer(s) as the light emitting proximal section. The proximal section 35 of the inner tubular member 20 formed of the light emitting polymeric blend is integrally formed with the distally adjacent section of the inner tubular member 20, as for example using an extrusion process in which the light emitting substance is present in only part of the extrudate. Alternatively, the proximal section 35 of the inner tubular member 20 formed of the light emitting polymeric blend is separately formed and then bonded to the distally adjacent section of the inner tubular member.

In the embodiment of FIG. 1, the light emitting substance is present in a wall section extending fully around the circumference of the inner tubular member 20. In alternative embodiments (not shown), the guidewire proximal port 23 is alternatively or additionally outlined by light emitting substance in or on the polymeric wall of the outer tubular member 19 around the port 23. For example, in one embodiment (not shown), the light emitting substance is along a shaft section 40 (see FIG. 1) commonly referred to as the rapid exchange catheter notch section, extending longitudinally at the guidewire proximal port 23. The length of notch section 40 of the shaft 11 having the light emitting substance can be longer or shorter than the length illustrated by dashed lines in the embodiment of FIG. 1, and is generally about 2 to about 3 cm, or about 1% to about 5% of the total length of the catheter shaft 11. Thus, a section of the outer tubular member 19, and optionally also the inner tubular member 20, at the guidewire proximal port 23 and extending fully around the circumference of the shaft 11, has the light emitting substance in one embodiment, to highlight the section of the shaft 11 having the guidewire proximal port 23 therein.

The rapid exchange catheter proximal adapter 28 connected to the shaft 11 in the embodiment of FIG. 1 has a distal end configured for connecting to a proximal end of the catheter shaft 11, and a proximal luer-type connector 42 defining a port 43 and configured for attaching to an inflation fluid source (not shown). The port 43 is in fluid communication with lumen 44 in the proximal adapter 28, which is in fluid communication with the inflation lumen 15 of the catheter 10. In the embodiment of FIG. 1, the rapid exchange catheter proximal adapter 28 has a polymeric wall with a light emitting substance 32, and specifically, is formed of a light emitting polymeric blend so that the proximal end face 45 of the adapter defining the port 43 is formed of the light emitting polymeric blend to thereby outline the port 43. As discussed above, the light emitting substance 32 can alternatively be applied as a coating on a surface of the adapter 28, such as an outer surface, inner surface, or distal end-face surface. At least the proximal luer-type connector 42 defining proximal port 43 of the adapter 28 has the light emitting substance, to outline the port 43 and facilitate viewing the port during connection to the source of inflation fluid. However, in one embodiment, the entire proximal adapter 28 is formed of a light emitting polymeric blend, typically by injection molding. A variety of suitable polymeric materials are used to form proximal adapter 28, and in a preferred embodiment the polymeric material is a polycarbonate.

The same or a different light emitting substance 32 is used to form the various light emitting components of the catheter 10. The preferred amount of light emitting substance in the polymeric blend will depend on factors such as the nature of the light emitting substance. In one embodiment, the light emitting substance is generally about 1 to about 33% by weight of the polymeric blend. In an embodiment in which the light emitting substance is applied as a coating on a surface of the polymeric wall defining a port, the light emitting substance is preferably blended with a matrix material such as a polymer or a liquid solvent to facilitate applying it as a coating. Preferably, the light emitting substance emits light in the cath-lab after activation (absorbing/being excited by) by the ambient visible light, including incandescent or fluorescent light, present in the cath-lab during the medical procedure. In one embodiment, the light emission lasts a period of time (e.g., on the order of minute(s)) after being activated and then isolated from the excitation source, with the light emission not continuing long after the light emitting component is introduced into the patient's body lumen.

FIG. 5 illustrates an alternative distal tip member 50, having an outer coating forming stripes 52 of light emitting substance on an outer surface of the polymeric wall of the distal tip member 50. The stripes 52 extend along a distal portion of the distal tip member 50 although they may alternatively extend the entire length. As best shown in FIG. 6 illustrating a transverse cross sectional view of the distal tip member 50 of FIG. 5 taken along line 6-6, the stripes 52 are spaced apart around the circumference of the distal tip member 50. The light emitting substance can be applied to partially coat the component in a variety of suitable configurations including circumferentially or spirally extending band(s). In the embodiments of FIGS. 1 and 5, the light emitting substance 32 is in or on an exposed outer surface of the distal tip member 30, 50, located distal to the distal end of the balloon 14 and extending to the distal end face of the distal tip member 30, 50, with at least part of the distal end face having the light emitting substance.

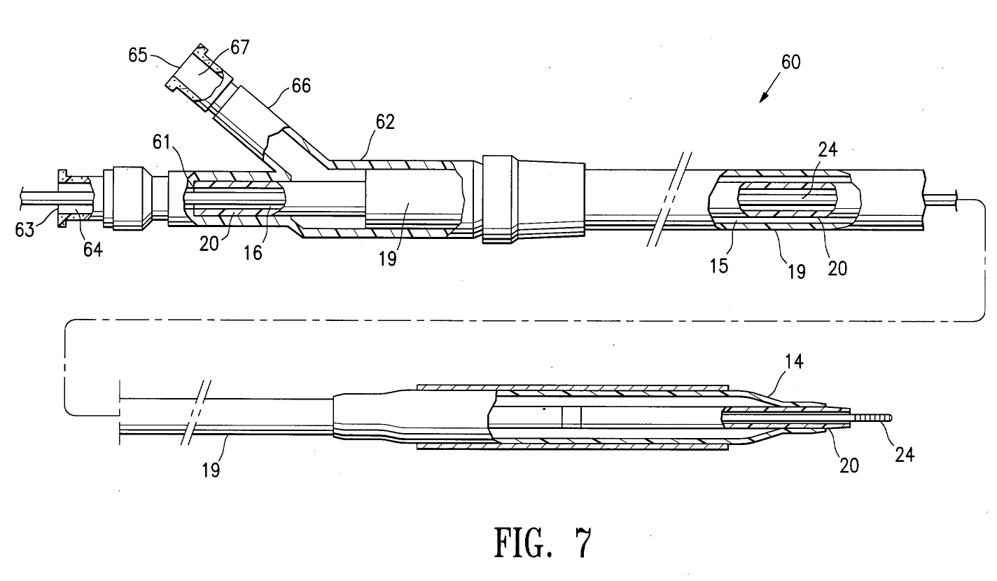

FIG. 7 illustrates an alternative catheter 60 embodying features of the invention. Catheter 60 is an over-the-wire type balloon catheter which, unlike the rapid exchange type balloon catheter 10 of FIG. 1, has the inner tubular member 20 proximal end 61 at the proximal end of the shaft 11, and a proximal Y-adapter 62 on the proximal end of the catheter shaft 11. The proximal adapter 62 has a distal end configured for connecting to a proximal end of a catheter, a first proximal port 63 configured to provide access to guidewire lumen 16 of inner tubular member 20 through lumen 64, and a second proximal port 65 in arm 66 configured for connecting to a source of inflation fluid (not shown) to direct inflation fluid into inflation lumen 15 through lumen 67. At least a section of the adapter defining at least one of the first and second proximal ports 63, 65 is formed at least in part by a light emitting substance 32, so that the at least one proximal port is outlined by the light emitting substance. As discussed above in relation to the embodiment of FIG. 1, the polymeric wall of the adapter 62 defining a lumen and a port is formed of a blend of a polymeric material and the light emitting substance 32, or alternatively has the light emitting substance 32 coated on an outer and/or an inner surface of the polymeric wall. In the illustrated embodiment, both the first and second proximal ports 63, 65 are outlined by the light emitting substance. In one embodiment, the entire proximal adapter 62 is formed of a light emitting polymeric blend. The preferred polymeric materials discussed above in relation to the formation of adapter 28 are similarly used to form adapter 62.

The dimensions of catheters 10/60 are determined largely by the size of the balloon and guidewire to be employed, the catheter type, and the size of the artery or other body lumen through which the catheter must pass or the size of the stent being delivered. Typically, the outer tubular member 19 has an outer diameter of about 0.025 to about 0.04 inch (0.064 to 0.10 cm), usually about 0.037 inch (0.094 cm), and the wall thickness of the outer tubular member 19 can vary from about 0.002 to about 0.008 inch (0.0051 to 0.02 cm), typically about 0.003 to about 0.005 inch (0.0076 to 0.013 cm). The inner tubular member 20 typically has an inner diameter of about 0.01 to about 0.018 inch (0.025 to 0.046 cm), usually about 0.016 inch (0.04 cm), and a wall thickness of about 0.004 to about 0.008 inch (0.01 to 0.02 cm). The overall length of the catheter 10 may range from about 100 to about 150 cm, and is typically about 143 cm. Preferably, balloon 14 has a length about 0.8 cm to about 6 cm, and an inflated working diameter of about 2 to about 10 mm.

Inner tubular member 20 and outer tubular member 19 can be formed by conventional techniques, for example by extruding and necking materials already found useful in intravascular catheters such a polyethylene, polyvinyl chloride, polyesters, polyamides, copolyamides, polyimides, polyurethanes, and composite materials. The various components may be joined using conventional bonding methods such as by fusion bonding or use of adhesives. In the embodiment illustrated in FIG. 1, the outer and inner tubular members 19, 20 are each formed of a single-layered, uniform polymeric member. However, it should be understood that in alternative embodiments, one or both of the outer and inner tubular members 19, 20 may be a multilayered or blended polymeric member. Although the shaft 11 is illustrated as having an inner and outer tubular member, a variety of suitable shaft configurations may be used including a dual lumen extruded shaft having a side-by-side lumens extruded therein.

While the present invention is described herein in terms of certain preferred embodiments, those skilled in the art will recognize that various modifications and improvements may be made to the invention without departing from the scope thereof. Moreover, although individual features of one embodiment of the invention may be discussed herein or shown in the drawings of the one embodiment and not in other embodiments, it should be apparent that individual features of one embodiment may be combined with one or more features of another embodiment or features from a plurality of embodiments.

What is claimed:

1. A catheter component, comprising a light emitting substance in or on at least a section of a polymeric wall defining a lumen and a port, so that the port is outlined by the light emitting substance.

2. The catheter component of claim 1, wherein the component is selected from the group consisting of a shaft, and a proximal adapter.

3. The catheter component of claim 1, wherein the component has an outer surface, and an inner surface defining the lumen therein, and the polymeric wall of the component has a width extending from the outer surface to the inner surface thereof, and the entire width of the at least a section of the polymeric wall is formed of a blend of a polymeric material and the light emitting substance.

4. The catheter component of claim 1, wherein the catheter is a balloon catheter and the component is a shaft having a distal end section extending distally beyond a distal end of the balloon, and the port is at a distal end of the distal end section of the shaft.

5. The catheter component of claim 4, wherein the distal end section of the shaft comprises a tubular distal tip member, and a distal end face of a tubular distal tip member has the port therein with at least part of the tubular distal tip member having the light emitting substance.

6. The catheter component of claim 5 wherein at least a distal portion of the tubular distal tip member is formed of a blend of a polymeric material and the light emitting substance, the blend defining the entire distal end face of the distal tip member from an outer surface to an inner surface of the distal tip member.

7. The catheter component of claim 1 wherein the light emitting substance is selected from the group consisting of a luminescent, a fluorescent, and a phosphorescent substance.

8. The catheter of claim 7 wherein the light emitting substance emits light having a wavelength of about 400 nm to 700 nm upon exposure to an energy source.

9. The catheter of claim 8 wherein the energy source is ambient light having a wavelength of about 400 nm to about 700 nm.

10. The catheter of claim 1 wherein the light emitting substance is a phosphorescent substance selected from the group consisting of zinc sulfide and strontium aluminate.

11. A balloon catheter, comprising:
a) an elongated shaft having a proximal end, a distal end, a distal shaft section, an inflation lumen, and a guidewire lumen in communication with a guidewire proximal port and a guidewire distal port, a polymeric wall defining the guidewire lumen at either or both of the guidewire proximal and distal ports having at least a section thereof with a light emitting substance in the polymeric wall; and
b) a balloon on the distal shaft section, having an interior in fluid communication with the inflation lumen.

12. The balloon catheter of claim 11 wherein the catheter shaft comprises a tubular member extending at least through the balloon interior and defining a first section of the guidewire lumen, and a distal tip member defining a second section of the guidewire lumen in communication with the first section of the guidewire lumen and defining the guidewire distal port.

13. The balloon catheter of claim 12 wherein at least part of the distal tip member is formed of a blend of a polymeric material and the light emitting substance.

14. The balloon catheter of claim 12 wherein the balloon has a distal skirt section secured to the catheter shaft and forming the distal end of the balloon, and at least part of the distal tip member extends distally beyond the distal end of the balloon.

15. The balloon catheter of claim 11 wherein the catheter shaft comprises an outer tubular member defining the inflation lumen and an inner tubular member defining at least a section of the guidewire lumen, the inner tubular member having at least a layer formed of a blend of a polymeric material and the light emitting substance.

16. The balloon catheter of claim 11 including a proximal adapter on the proximal end of the elongated shaft, comprising a distal end configured for connecting to the proximal end of the catheter, and at least one proximal port, at least a section of the adapter defining the at least one proximal port is formed at least in part by a light emitting substance.

17. A proximal adapter for a catheter, comprising a distal end configured for connecting to a proximal end of a catheter, and at least one proximal port, at least a section of the adapter defining the at least one proximal port is formed at least in part by a light emitting substance, so that the at least one proximal port is outlined by the light emitting substance.

18. The proximal adapter of claim 17 wherein the at least a section of the adapter formed at least in part by the light emitting substance comprises a polymeric wall which defines a lumen and which is formed of a blend of a polymeric material and the light emitting substance.

19. The proximal adapter of claim 17 wherein the at least a section of the adapter formed at least in part by the light emitting substance comprises a polymeric wall which defines a lumen and which has the light emitting substance coated on an outer and/or an inner surface of the polymeric wall.

20. A method of making a catheter component, comprising:
a) blending a polymeric material and a light emitting substance together to form a blend; and
b) extruding or molding the blend to form at least a section of a polymeric wall defining a port, so that the port is outlined by the light emitting substance.
